(19) United States
(12) Patent Application Publication
(10) Pub. No.: US 2024/0041048 A1
(43) Pub. Date: Feb. 8, 2024

(54) NUTRIENT AND INNOCULANT COMPOSITION AND METHOD OF USING

(71) Applicant: KOCH AGRONOMIC SERVICES, LLC, Wichita, KS (US)

(72) Inventors: Sara Segovia, Overland Park, KS (US); Ithamar Prada, Sao Paulo (BR); Ryan Bartlett, Basehor, KS (US); Renil John Anthony, Lenexa, KS (US); Robert A. Geiger, Denton, TX (US); Dinesh Adhikari, Cary, NC (US)

(21) Appl. No.: 18/268,617

(22) PCT Filed: Dec. 20, 2021
(86) PCT No.: PCT/IB2021/062040

§ 371 Date: Jun. 21, 2023

Related U.S. Application Data
(60) Provisional application No. 63/129,044, filed on Dec. 22, 2020.

(51) Int. Cl. A01N 63/20; A01H 5/10

(52) U.S. Cl. CPC A01N 63/20; A01H 5/10

(57) ABSTRACT

The present disclosure provides a new composition for use as a nutrient and soybean inoculant. The composition comprises an inoculant (e.g., Bradyrhizobium), one or more plant nutrients, and preferably a carrier for the inoculant and one or more plant nutrients. Additionally, the composition is preferably free of peat and advantageously the Bradyrhizobia are not harmed and remain active, even when stored with the nutrients. The composition can be applied to soil and/or to soybean seeds, thus assisting soybean seeds in fixing nitrogen.

### CROSS REFERENCES TO RELATED APPLICATIONS

This application claims priority from U.S. Provisional Patent Application No. 63/129,044, filed Dec. 22, 2020, which is hereby incorporated by reference in its entirety.

### FIELD OF THE DISCLOSURE

The present disclosure broadly relates to a nutrient and inoculant composition and a method of using that composition.

### BACKGROUND OF THE DISCLOSURE

Unlike many other crops, soybeans are able to obtain their own nitrogen via nitrogen fixation by taking advantage of a symbiotic relationship with Bradyrhizobium, and particularly with the species Bradyrhizobium japonicum and Bradyrhizobia elkanii. The Bradyrhizobia invade the root hairs of the soybean plants, forming nodules on the plant roots. These nodules fix nitrogen from the atmosphere and supply it to the soybean plants. The soybean plants reciprocate by providing a carbohydrate supply to the bacteria, thus allowing the bacteria to thrive as well.

This process usually allows farmers to avoid costly nitrogen fertilization. However, in some soils there may be an insufficiency of Bradyrhizobia, depending upon a number of factors. For example, if soybeans have not been planted in several years, the bacteria may be lacking in the soil. In the instances of insufficient bacteria, soybeans can benefit from inoculation with the bacteria. In North America, an inoculant is used on approximately 45% of soybean acres. While there is a substantial advantage to inoculation, it can be costly, there are compatibility issues with other treatments, and application at the retailer often brings complexities.

A significant problem with current method of inoculating soybean seeds is that the inoculant and nutrients cannot be stored together as the bacteria are harmed by the nutrients and thus become less effective. Because of this, the inoculant must be applied separately from any nutrients as the nutrients are harmful to the inoculant and negatively affect the inoculant's viability on the seed. This results in a more expensive and time-consuming process.

There is a need for a composition that avoids the problems of existing inoculant products and preferably also simplifies the application process for the user.

### SUMMARY OF THE DISCLOSURE

The present disclosure is broadly directed towards an inoculation method comprising introducing a composition into an environment and/or contacting a seed with the composition. The composition comprises an inoculant and a plant nutrient.

In one embodiment, a seed having an outer surface and comprising a composition on at least some of that outer surface is provided. The composition comprises an inoculant and a plant nutrient.

The disclosure also provides an inoculation composition comprising an inoculant, a plant nutrient, and a carrier for the inoculant and the plant nutrient.

A mixture comprising soil, an inoculant, a plant nutrient, and a carrier for the inoculant and plant nutrient is also disclosed herein.

### BRIEF DESCRIPTION OF THE FIGURES

FIG. 1 illustrates that the psyllium released the copper back into solution, indicating that once the materials are combined, they are again water soluble.

FIG. 2(A): top left tray contains pellet #4; top right tray contains pellet #5; bottom left tray contains pellet #3; and bottom right tray contains pellet #2.

FIG. 2(B): top left petri dish is a dilution of one pellet #4; top right petri dish is a dilution of one pellet #4; bottom left petri dish is a dilution of one pellet #5; and bottom right petri dish is a dilution of one pellet #5.

FIG. 3 is a graph showing the average nodule count by seed treatment after the 4-week soybean growth period.

### DETAILED DESCRIPTION OF THE DISCLOSURE

### Inoculant Compositions

The compositions according to the disclosure generally comprise an inoculant and a plant nutrient(s). Even more preferably, the compositions comprise a carrier for the inoculant and plant nutrient(s).

The carrier should be one that can disperse the inoculant and plant nutrient and is also inert. “Inert” as used herein means that the carrier does not have a negative impact on the inoculant or the nutrient. For example, the carrier can remain in contact with the inoculant and/or plant nutrient for extended periods of time (e.g., 3 months or more, or even 6 months or more) without killing the inoculant and preferably without degrading or decomposing either of the inoculant or the plant nutrient. Carriers suitable for use in the compositions preferably comprise complex carbohydrates, such as polysaccharides. Fibrous materials such as cellulosic materials are particularly suitable. It is also preferred that the carrier is a natural material and more preferably derived from a plant such as vegetable or grain plants. Some preferred carriers for use in the compositions according to the disclosure are husks (e.g., grain or cereal husks, vegetable husks) and/or hulls. Examples of suitable carriers include husks and/or hulls from one or more of psyllium, grain, corn (maize), wheat, rice, barley, oats, rye, sorghum, soybeans, and hemp. Other carriers that can be used in some embodiments include seaweed, humic substances, peat moss, and mixtures thereof.

Another suitable carrier for use in the inoculant compositions comprises ground corn components. Suitable ground corn components include, but are not limited to, those selected from the group consisting of cornmeal (which encompasses corn flour), corn starch, and mixtures thereof. In one embodiment, a mixture of corn starch and cornmeal is utilized. It is preferred that the starch present in the ground corn components has not been modified in any way. For example, it is preferred that the starch molecules have not been grafted or otherwise reacted with any compounds or polymers (particularly non-starch polymers).

The carrier could also comprise a vegetable starch such as starches selected from the group consisting of potato starch, pea starch, sweet potato starch, bean starch, chickpea starch, squash starch, yam starch, and mixtures thereof. Other acceptable starches include cereal starches such as those selected from the group consisting of wheat starch, rice starch, tapioca starch, rye starch, oat starch, barley starch, sorghum starch, and mixtures thereof. These other starches are also preferably unmodified, as discussed previously with respect to the corn starch present in the ground corn components.

The carrier is utilized in amounts of about 10% to about 60% by weight carrier, preferably from about 20% to about 50% by weight carrier, and more preferably from about 25% to about 45% by weight carrier, based on the total weight of the composition taken as 100% by weight.

The preferred inoculant is a nitrogen-fixing organism such as Bradyrhizobium (and preferably Bradyrhizobium japonicum and/or Bradyrhizobia elkanii). The inoculant is preferably present at levels of about 1×10² CFU/g to about 1×10⁹ CFU/g of composition, more preferably about 1×10³ CFU/g to about 1×10⁷ CFU/g of composition, and even more preferably about 1×10³ CFU/g to about 1×10⁶ CFU/g of composition.

In a preferred embodiment, the plant nutrient of the composition is selected from the group consisting of sources of macronutrients, micronutrients, and mixtures thereof. As used herein, “macronutrient” refers to elements typically required in large quantities for plant growth, with preferred macronutrients being those selected from the group consisting of calcium, sulfur, phosphorus, magnesium, potassium, nitrogen, sodium, and mixtures thereof. “Micronutrient” refers to elements typically required in small or trace amounts for plant growth, with preferred macronutrients being those selected from the group consisting of nickel, copper, zinc, manganese, boron, iron, cobalt, selenium, molybdenum, and mixtures thereof. In both instances, a “source” of a macronutrient or micronutrient is meant to refer to a compound containing the element (e.g., Cu-EDTA) or the element itself (e.g., Cu), unless stated otherwise.

It will be appreciated that the respective quantities of macronutrient sources and/or micronutrient sources can be adjusted depending upon crop species, soil conditions, environmental factors, etc., but it is generally preferred that the overall total quantity of all macronutrient and micronutrient sources in the composition is from about 10% to about 70% by weight, preferably from about 10% to about 55% by weight, and more preferably from about 30% by weight to about 50% by weight, based upon the total weight of the composition taken as 100% by weight.

Preferred quantities of typical macronutrients and micronutrients are shown in Table A.

TABLE A

| PLANT | BROADEST | | MOST |
|---|---|---|---|
| NUTRIENT* | RANGE** | PREFERRED* | PREFERRED** |
| Calcium (Ca) | about 0.001% to about | about 0.01% to about | about 0.01% to about |
| | 45% | 25% | 15% |
| Sulfur (S) | about 0.001% to about | about 0.01% to about | about 0.01% to about |
| | 45% | 25% | 15% |
| Magnesium (Mg) | about 0.001% to about | about 0.01% to about | about 0.01% to about |
| | 45% | 25% | 15% |
| Potassium (K) | about 0.001% to about | about 0.01% to about | about 0.01% to about |
| | 45% | 25% | 15% |
| Nitrogen (N) | about 0.001% to about | about 0.01% to about | about 0.01% to about |
| | 45% | 25% | 15% |
| Sodium (Na) | about 0.001% to about | about 0.01% to about | about 0.01% to about |
| | 45% | 25% | 15% |
| Nickel (Ni) | about 0.001% to about | about 0.01 to about 25% | about 0.01% to about |
| | 45% | | 15% |
| Copper (Cu) | about 0.001% to about | about 0.01% to about | about 0.01% to about |
| | 45% | 25% | 15% |
| Zinc (Zn) | about 0.001% to about | about 0.01% to about | about 0.01% to about |
| | 45% | 25% | 15% |
| Manganese (Mn) | about 0.001% to about | about 0.01% to about | about 0.01% to about |
| | 45% | 25% | 15% |
| Boron (B) | about 0.001% to about | about 0.01% to about | about 0.01% to about |
| | 45% | 25% | 15% |
| Iron (Fe) | about 0.001% to about | about 0.01% to about | about 0.01% to about |
| | 45% | 25% | 15% |
| Cobalt (Co) | about 0.001% to about | about 0.01% to about | about 0.01% to about |
| | 45% | 25% | 15% |
| Selenium (Se) | about 0.001% to about | about 0.01% to about | about 0.01% to about |
| | 45% | 25% | 15% |
| Molybdenum (Mo) | about 0.001% to about | about 0.01 to about 25% | about 0.01% to about |
| | 45% | | 15% |

*In embodiments where the particular nutrient is present (i.e., when it is not 0%).
**All ranges refer to the weight of the nutrient rather than the source of the nutrient, with % by weight being based upon the total weight of the composition taken as 100% by weight.

In one embodiment, the carrier is a carrier other than peat and/or humic substances. That is, the composition comprises limited quantities of peat and/or humic substances or is even essentially free of peat and/or humic substances. In this embodiment, the composition preferably comprises less than about 50% by weight peat and/or humic substances, more preferably less than about 30% by weight peat and/or humic substances, even more preferably less than about 20% by weight peat and/or humic substances, most preferably less than about 5% by weight peat and/or humic substances, and most preferably about 0% by weight peat and/or humic substances, based on the total weight of the composition taken as 100% by weight. “Peat” forms when plant material does not fully decay in acidic and anaerobic conditions. Thus, peat comprises partially decayed vegetation and/or plant (humus) matter. Some components in peat include decayed and/or partially decayed Sphagnum moss (also called “peat moss”), sedges and/or grasses (e.g., Carex, reed grass, Phragmites australis), shrubs, and/or other acid-loving plants. Peat also tends to include humic acids and/or fulvic acids.

In one embodiment, it is preferred that the composition is essentially free of one, two, three, or four of the following: waxes, carbonaceous materials (e.g., graphite or other materials whose weight is at least 90% attributable to carbon), silicon-containing compounds (e.g., silicates such as talc, clays such as montmorillonite, kaolinite, and bentonite), microorganisms other than the inoculant (e.g., Bradyrhizobium), and polymers other than those naturally present in the carrier. In these such embodiments, the composition comprises less than about 5% by weight total, preferably less than about 3% by weight total, and more preferably about 0% by weight total of one, two, three, or four of the foregoing, based upon the total weight of the composition taken as 100% by weight.

Even more preferably, the composition is essentially free of all five of waxes, carbonaceous materials, silicon-containing compounds, microorganisms other than the inoculant (e.g., Bradyrhizobium), and polymers other than those naturally present in the carrier. Thus, the cumulative total of the foregoing is less than about 5% by weight, preferably less than about 3% by weight, and more preferably about 0% by weight, based upon the total weight of the composition taken as 100% by weight.

In one embodiment, the composition consists essentially of, or even consists of, the inoculant, the carrier, and one or more plant nutrients.

In one embodiment, the composition consists essentially of, or even consists of, Bradyrhizobium, the carrier, and one or more plant nutrients.

In another embodiment, the composition consists essentially of, or even consists of, the inoculant, ground corn components, and one or more plant nutrients.

In a further embodiment, the composition consists essentially of, or even consists of, Bradyrhizobium, ground corn components, and one or more plant nutrients.

In an alternative embodiment, the composition further comprises mica, and more preferably mica that is coated with TiO₂. The inoculant, carrier, and plant nutrient(s) are preferably present in the ranges discussed previously. It is preferred that the TiO₂-coated mica is present at levels of from about 0.4% to about 25% by weight, preferably from about 1% to about 10% by weight, and more preferably from about 1.5% by weight to about 5% by weight, based upon the total weight of the composition taken as 100% by weight. Additionally, a dye or colorant is optionally included, and when it is included, it is present at levels of from about 0.1% to about 15% by weight, preferably from about 1% to about 10% by weight, and more preferably from about 1% by weight to about 5% by weight, based upon the total weight of the composition taken as 100% by weight.

In a further embodiment, the composition consists essentially of, or even consists of, inoculant, carrier, plant nutrient(s), TiO₂-coated mica, and optionally a dye or colorant.

In another embodiment, optional ingredients can be added, such as those selected from the group consisting of biostimulants, microorganisms, dispersants, other inoculants, and/or anti-caking agents.

Advantageously, each ingredient utilized to form the composition is provided in powder or particulate form. The average particle size of each ingredient utilized should be less than about 175 μm, preferably from about 25 μm to about 175 μm, and more preferably from about 100 μm to about 160 μm. In one embodiment, at least about 50%, preferably at least about 70%, more preferably at least about 85%, even more preferably at least about 95%, and most preferably about 100% of the particles in the fertilizer composition will have a particle size in this range. The particle size is determined by conventional methods, including by simply passing the particles through an analytical sieve to screen out particles having an undesirable size. Additionally, the ingredients can be individually subjected to a particular size reduction process (e.g., milling) to achieve these sizes, or the formulation can be prepared, followed by particle size reduction of the entire formulation.

It is preferred that the compositions are provided in a dry, particulate form. That is, the composition will have a moisture content of less than about 5% by weight, preferably less than about 3% by weight, more preferably less than about 1% by weight, and preferably about 0% by weight, based upon the total weight of the composition taken as 100% by weight. These levels can be achieved by providing the individual ingredients in a substantially dry form or by drying the final composition to these levels.

Finally, the compositions are prepared by simply blending the ingredients described above to form a substantially homogenous mixture. As noted above, these ingredients are subjected to particle size reduction prior to blending, as needed. Alternatively, or additionally, particle size reduction of the final mixture can be carried out after it is prepared.

### Methods of Using Compositions

The method of using the inventive compositions comprises contacting a seed (preferably a nitrogen-fixing crop species, such as a soybean seed) or plurality of seeds with the composition so that the composition coats at least some of the outer surface of each seed, and preferably coats the majority of the respective outer surfaces of the seeds. That is, the average outer surface is at least about 50% coated, preferably at least about 75% coated, more preferably at least about 90% coated, and even more preferably about 100% coated with the composition. This contacting preferably occurs before contact of the seed with soil so that the seeds are coated with the composition prior to planting.

It will be appreciated that the application rate can be adjusted as deemed necessary for the particular seed and other conditions. Typically, this results in an application rate of from about 0.2 grams to about 4 grams per kg of seed, preferably from about 1 to about 4 grams per kg of seed, and more preferably from about 2 to about 4 grams per kg seed. Alternatively, the rate would be from about 0.02% by weight to about 0.4% by weight, preferably from about 0.1% by weight to about 0.4% by weight, and more preferably from about 0.2% by weight to about 0.4% by weight, based upon the total weight of the seed taken as 100% by weight.

This process can be carried out by any conventional seed-coating process, including using a hopper box, planter box, batch seed treater, or blender. Additionally, the composition can be applied to dry seeds or wet seeds, and the coated seeds can be planted following conventional planting processes. This can take place immediately after coating, or the coated seeds can be stored for planting at a later date.

Alternatively, the composition can be introduced into soil where soybean seeds have been, or will be, planted.

Regardless of the application method, the present composition offers a significant advantage over the prior art in that the inoculant is not harmed by the presence of nutrients in the same composition as the inoculant. Thus, the nutrients and nitrogen-fixing organism can be present in the same composition and stored together until use without negatively impacting the activity of the nitrogen-fixing organism. That is, at about 60 days, 100 day, or 365 days (or even longer) after the composition has been formed, at least about 75%, preferably at least about 85%, and more preferably from about 90% to about 100% of the original CFUs of Bradyrhizobium remain viable. It will be appreciated that this is significant because it eliminates an entire application step by avoiding the need for separate nutrient and inoculant application steps. Additionally, using the inoculant compositions as described herein results in increased nodule formation, root biomass, and root hairs as well as enhanced enzymatic biosynthesis for nodule development.

Additional advantages of the various embodiments of the disclosure will be apparent to those skilled in the art upon review of the disclosure herein and the working Examples below. It will be appreciated that the various embodiments described herein are not necessarily mutually exclusive unless otherwise indicated herein. For example, a feature described or depicted in one embodiment may also be included in other embodiments but is not necessarily included. Thus, the present disclosure encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

All ranges provided herein include each and every value in the range as well as all sub-ranges there-in-between as if each such value or sub-range were disclosed. Additionally, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

Further, all aspects and embodiments of the disclosure comprise, consist essentially of, or consist of any aspect or embodiment, or combination of aspects and embodiments disclosed herein.

### EXAMPLES

The following examples set forth methods in accordance with the disclosure. It is to be understood, however, that these examples are provided by way of illustration and nothing therein should be taken as a limitation upon the overall scope of the disclosure.

### Example 1

### 1. Psyllium Test

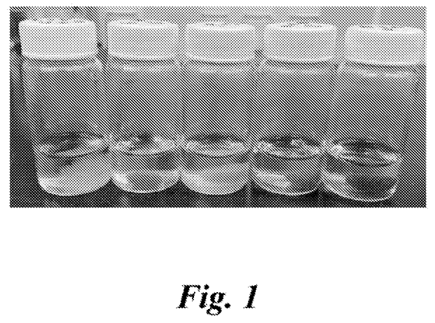

This test was carried out to determine if psyllium would release a substance after it has absorbed or “soaked up” that substance. Pellets #1-5 were formed using the components described below with a water dropper containing 0.4% mannitol and 99.6% water. The pellets were sieved and dried at 32° C. in an oven overnight. The pellets were added to water for observation. The vials are shown in FIG. 1 (left to right):
1. 33.33% by weight Cu-EDTA+66.66% by weight psyllium husk powder (to determine visually whether the pellet would release soluble ingredients (Cu-EDTA) into the water or hold the soluble ingredients in the pellet);
2. 66.6% by weight psyllium husk powder+33.33% by weight ROCKET SEEDS® Moly Dry 1-5-0 (nutrients and ground corn components; obtained from Compass Minerals Plant Nutrition);
3. 100% by weight psyllium husk;
4. 16.66% by weight ROCKET SEEDS® Moly Dry+66.6% by weight psyllium husk powder+16.66% by weight Bradyrhizobia japonicum (TerraMax Dry; obtained from TerraMax, Inc.); and
5. 66.6% by weight psyllium husk powder+33.33% by weight Bradyrhizobia japonicum.

These results show that psyllium released the copper back into solution, indicating that once the materials are combined, they are again water soluble.

### 2. Bradyrhizobia Growth

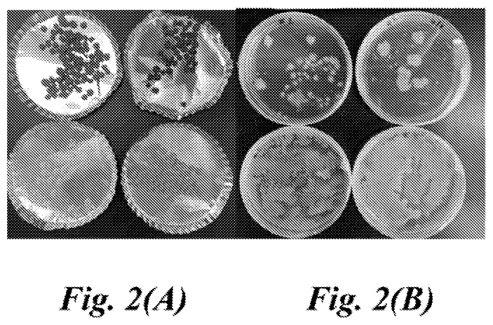

Pellets #2-5 from Part 1 of this Example were plated on agar to determine whether Bradyrhizobia japonicum would grow in the presence of psyllium and nutrients. These results confirmed that growth was observed in the presence of psyllium and the nutrient composition, even when a lower concentration of Bradyrhizobia japonicum was included in the pellet. The results are illustrated in FIG. 2(A) and FIG. 2(B).

### Example 2

Bradyrhizobium japonicum was combined with ROCKET SEEDS® Moly Dry or ROCKET SEEDS® Moly Liquid to determine whether either product had a deleterious effect on the Bradyrhizobium japonicum. The CFU/g of each sample was determined at 0, 7, and 14 days by taking a known amount of the particular product and preparing a solution dilution series with water/buffer. A known amount of the diluted solution was spread onto a plate, and the colonies that grew on the plate were counted. That number was used to calculate the CFU per gram of product using the known original amount, dilution factor, and final amount applied to the plate. These results (Table 1) showed that at 14 days after combination, the Bradyrhizobium japonicum with ROCKET SEEDS® Moly Dry as the nutrient component remained alive compared to the Bradyrhizobium japonicum with ROCKET SEEDS® Moly Liquid (obtained from Compass Minerals Plant Nutrition) as the nutrient component. It is hypothesized that the ROCKET SEEDS® Moly Liquid formula “suffocated” the Bradyrhizobium japonicum and/or did not have buffering capabilities to reduce any harm from the salts, metals, and other ingredients used to hold the suspension.

TABLE 1

| | CFU/g. | CFU/g. | CFU/g. |
|---|---|---|---|
| SAMPLE | 0 days | 7 days | 14 days |
| Prototype (ROCKET SEEDS ® Moly Dry + | 3.90E+05 | 1.20E+05 | 2.60E+05 |
| Bradyrhizobium japonicum) | | | |
| Bradyrhizobium japonicum (Dry source) | 1.80E+06 | 2.20E+06 | 2.40E+06 |
| ROCKET SEEDS ® Moly Dry* | 1.00E+02 | <100 | <100 |
| ROCKET SEEDS ® Moly Liquid + Bradyrhizobium | 6.00E+03 | 1.40E+03 | <100 |
| japonicum | | | |
| Bradyrhizobium japonicum (peat-based liquid source) | 1.00E+06 | 5.10E+05 | 6.60E+05 |
| ROCKET SEEDS ® Moly Liquid | <100 | <100 | <100 |
| Control** | <100 | <100 | <100 |

*It is suspected that the corn meal present in the nutrient component contained some microbes.
**Water/buffer solution for preparing dilution series.

### Example 3

### Soybean Nodulation

This agronomic trial was carried out in a greenhouse to demonstrate increased efficacy of a Bradyrhizobia and nutrient composition on soybean nodulation and soybean biomass. Seeds were treated with a nutrient component (ROCKET SEEDS® Moly Dry) and Bradyrhizobia japonicum (TerraMax Dry). The seed treatments comprised:
1. Untreated soybean seed;
2. Bradyrhizobia japonicum applied to seed at a rate of 2 oz/50 lb seed (56.7 g/22.7 kg seed);
3. Nutrient component alone applied to seed at a rate of 1.5 oz/50 lb seed (42.5 g/22.7 kg seed); and
4. Combination of 2 and 3.

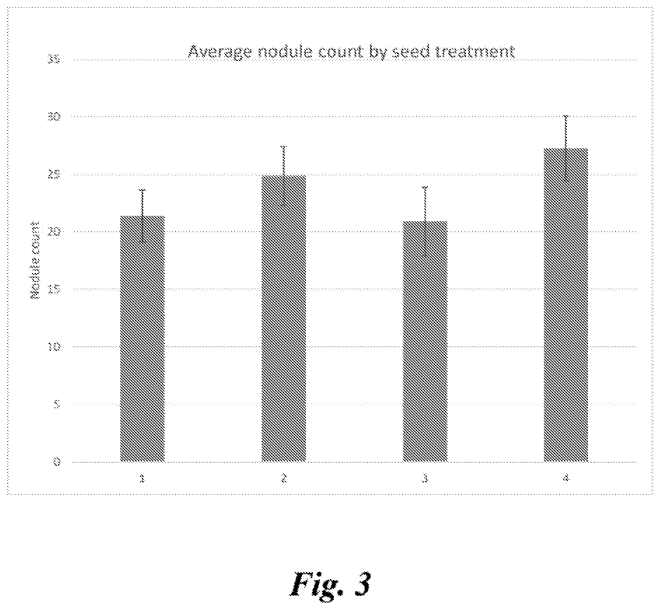

The treated seeds were grown in the greenhouse for 4 weeks and then harvested to observe root nodule formation. The average nodule count by seed treatment after the 4-week soybean growth period is shown in FIG. 3.

### Example 4

### Field Trial

This field trial was carried out to observe the efficacy of a Bradyrhizobia and nutrient composition on soybean nodulation and soybean biomass. Seeds were treated with a nutrient component (ROCKET SEEDS® Moly Dry) and Bradyrhizobia japonicum (TerraMax Dry). The seed treatments comprised:
1. Untreated soybean seed;
2. Bradyrhizobia japonicum applied to seed at a rate of 2 oz/50 lb seed (56.7 g/22.7 kg seed);
3. Nutrient component alone applied to seed at a rate of 1.5 oz/50 lb seed (42.5 g/22.7 kg seed); and
4. Combination of 2 and 3.

The treated seeds were planted in a field, grown for about 2 months, and then harvested to observe root nodule formation and determine dry biomass. Table 2 shows these results, which indicated that treatment #4 performed better on average than treatments #1, 2, or 3.

TABLE 2

| Treatment # | Rep. # | Trifoliate/plant | Dry biomass (g) | Nodules/plant |
|---|---|---|---|---|
| 1 | 1 | Branch1: 6, | 2.12 | 35 |
| | | Branch2: 6 | | |
| 1 | 2 | 9 | 1.672 | 39 |
| 1 | 3 | 8 | 2.313 | 40 |
| 2 | 1 | 18 | 5.343 | 73 |
| 2 | 2 | 19 | 5.717 | 88 |
| 2 | 3 | 14 | 3.047 | 42 |
| 2 | 4 | 11 | 0.827 | 22 |
| 3 | 1 | 14 | 3.899 | 65 |
| 3 | 2 | 13 | 2.631 | 47 |
| 3 | 3 | 16 | 4.19 | 34 |
| 3 | 4 | 21 | 7.389 | 50 |
| 4 | 1 | 20 | 6.765 | 66 |
| 4 | 2 | 17 | 6.897 | 59 |
| 4 | 3 | 16 | 4.498 | 61 |
| 4 | 4 | 25 | 6.773 | 64 |
| 4 | 5 | 15 | 3.529 | 62 |

Thus, using a treatment according to the disclosure will yield plants (about 2 months after planting) having an average dry biomass that is at least about 1.5 times, preferably at least about 2.0 times, more preferably at least about 2.5 times, and even more preferably from about 2.5 to about 5 times that of a plant grown from untreated seeds. Additionally, using a treatment according to the disclosure will yield plants having an average dry biomass that is at least about 1.1 times, preferably at least about 1.3 times, more preferably at least about 1.5 times, and even more preferably from about 1.5 to about 5 times that of a plant grown from seeds treated only with the same inoculant (i.e., without a nutrient). Using a treatment according to the disclosure will also yield plants having an average dry biomass that is at least about 1.1 times, preferably at least about 1.2 times, more preferably at least about 1.3 times, and even more preferably from about 1.3 to about 5 times that of a plant grown from seeds treated only with the same nutrients (i.e., without the inoculant).

In another embodiment, using a treatment according to the disclosure will yield plants (about 2 months after planting) having an average number of nodules per plant that is at least about 1.1 times, preferably at least about 1.3 times, more preferably at least about 1.5 times, and even more preferably from about 1.5 to about 5 times that of a plant grown from untreated seeds. Additionally, using a treatment according to the disclosure will yield plants having an average number of nodules per plant that is at least about 1.03 times, preferably at least about 1.05 times, more preferably at least about 1.1 times, and even more preferably from about 1.1 to about 5 times that of a plant grown from seeds treated only with the same inoculant (i.e., without a nutrient). Using a treatment according to the disclosure will also yield plants having an average number of nodules per plant that is at least about 1.05 times, preferably at least about 1.1 times, more preferably at least about 1.2 times, and even more preferably from about 1.2 to about 5 times that of a plant grown from seeds treated only with the same nutrients (i.e., without the inoculant).

1-49. (canceled)

50. An inoculation method comprising introducing a composition into an environment and/or contacting a seed with the composition, wherein the composition comprises an inoculant, a plant nutrient, and a carrier,
wherein the inoculant comprises Bradyrhizobium,
the plant nutrient is selected from the group consisting of sources of nickel, copper, zinc, manganese, boron, iron, chloride, selenium, molybdenum, calcium, sulfur, phosphorus, magnesium, potassium, nitrogen, sodium, and mixtures thereof, and
the carrier comprises ground corn components; and
wherein the composition is in a dry particulate form, wherein each component of the composition has an average particle size of less than about 175 μm and wherein the composition has a moisture content of less than about 5% by weight.

51. The inoculation method of claim 50, wherein the environment is soil and/or wherein the seed is a soybean seed.

52. The inoculation method of claim 50, further comprising contacting a plurality of the seeds and planting the seeds after contacting.

53. The inoculation method of claim 50, wherein the Bradyrhizobium comprises Bradyrhizobium japonicum, Bradyrhizobia elkanii, or a mixture thereof.

54. The inoculation method of claim 50, wherein the ground corn components are selected from the group consisting of corn starch, cornmeal, and mixtures thereof.

55. The inoculation method of claim 50, wherein the composition comprises less than about 30% by weight peat, based on the total weight of the composition taken as 100% by weight.

56. The inoculation method of claim 50, wherein the composition further comprises mica coated with TiO₂.

57. The inoculation method of claim 50, wherein the composition further comprises a dye or colorant.

58. The inoculation method of claim 50, wherein the inoculant is present at a level of about 1×10² CFU/g of the composition to about 1×10⁹ CFU/g of the composition, and
wherein the plant nutrient is present in an amount of about 10% to about 70% by weight, based on the total weight of the composition taken as 100% by weight, and
wherein the carrier is present in an amount of about 10% to about 60% by weight, based on the total weight of the composition taken as 100% by weight.

59. The inoculation method of claim 50, wherein the composition consists of an inoculant, a plant nutrient, and a carrier.

60. A seed having an outer surface and comprising a composition on at least some of the outer surface, wherein the composition comprises an inoculant, a plant nutrient, and a carrier, wherein
the inoculant comprises Bradyrhizobium,
the plant nutrient is selected from the group consisting of sources of nickel, copper, zinc, manganese, boron, iron, chloride, selenium, molybdenum, calcium, sulfur, phosphorus, magnesium, potassium, nitrogen, sodium, and mixtures thereof, and
the carrier comprises ground corn components; and
wherein the composition is in a dry particulate form, wherein each component of the composition has an average particle size of less than about 175 μm and wherein the composition has a moisture content of less than about 5% by weight.

61. The seed of claim 60, wherein the composition is on the majority of the outer surface.

62. The seed of claim 60, wherein the seed is a soybean seed.

63. The seed of claim 60, wherein the Bradyrhizobium comprises Bradyrhizobium japonicum, Bradyrhizobia elkanii, or a mixture thereof.

64. The seed of claim 60, wherein the ground corn components are selected from the group consisting of corn starch, cornmeal, and mixtures thereof.

65. The seed of claim 60, wherein the composition comprises less than about 30% by weight peat, based on the total weight of the composition taken as 100% by weight.

66. The seed of claim 60, wherein the composition further comprises mica coated with TiO₂.

67. The seed of claim 60, wherein the composition further comprises a dye or colorant.

68. The seed of claim 60, wherein the inoculant is present at a level of about 1×10² CFU/g of the composition to about 1×10⁹ CFU/g of the composition, and
wherein the plant nutrient is present in an amount of about 10% to about 70% by weight, based on the total weight of the composition taken as 100% by weight, and
wherein the carrier is present in an amount of about 10% to about 60% by weight, based on the total weight of the composition taken as 100% by weight.

69. The seed of claim 60, wherein the composition consists of an inoculant, a plant nutrient, and a carrier.

70. An inoculation composition comprising an inoculant, a plant nutrient, and a carrier, wherein
the inoculant comprises Bradyrhizobium,
the plant nutrient is selected from the group consisting of sources of nickel, copper, zinc, manganese, boron, iron, chloride, selenium, molybdenum, calcium, sulfur, phosphorus, magnesium, potassium, nitrogen, sodium, and mixtures thereof, and
the carrier comprises ground corn components; and
wherein the composition is in a dry particulate form, wherein each component of the composition has an average particle size of less than about 175 μm and wherein the composition has a moisture content of less than about 5% by weight.

71. The inoculation composition of claim 70, wherein the Bradyrhizobium comprises Bradyrhizobium japonicum, Bradyrhizobia elkanii, or a mixture thereof.

72. The inoculation composition of claim 70, wherein the ground corn components are selected from the group consisting of corn starch, cornmeal, and mixtures thereof.

73. The inoculation composition of claim 70, wherein the composition comprises less than about 30% by weight peat, based on the total weight of the composition taken as 100% by weight.

74. The inoculation composition of claim 70, wherein the composition further comprises mica coated with TiO₂.

75. The inoculation composition of claim 70, wherein the composition further comprises a dye or colorant.

76. The inoculation composition of claim 70, wherein the inoculant is present at a level of about 1×10² CFU/g of the composition to about 1×10⁹ CFU/g of the composition, and
wherein the plant nutrient is present in an amount of about 10% to about 70% by weight, based on the total weight of the composition taken as 100% by weight, and
wherein the carrier is present in an amount of about 10% to about 60% by weight, based on the total weight of the composition taken as 100% by weight.

77. The inoculation composition of claim 70, wherein the composition consists of an inoculant, a plant nutrient, and a carrier.
